(19) United States
(12) Patent Application Publication
(10) Pub. No.: US 2019/0015019 A1
(43) Pub. Date: Jan. 17, 2019

(54) MONITORING PLAYER PERFORMANCE

(71) Applicant: Intel Corporation, Santa Clara, CA (US)

(72) Inventors: James Brian Hall, Tucson, AZ (US); Tyler Fetters, Hillsboro, OR (US); Stephanie Moyerman, Phoenix, AZ (US); Jeffrey Mitsuo Ota, Morgan Hill, CA (US); Narayan Sundararajan, Palo Alto, CA (US)

(73) Assignee: Intel Corporation, Santa Clara, CA (US)

(21) Appl. No.: 16/065,390

(22) PCT Filed: Dec. 22, 2015
(86) PCT No.: PCT/US2015/067253

§ 371 Date: Jun. 22, 2018

(51) Int. Cl. A61B 5/11; A61B 5/00; G06F 15/18; H04W 4/80

(52) U.S. Cl. CPC A61B 5/1124; A61B 5/1122; A61B 5/6895; A61B 2503/10; H04W 4/80; A61B 2562/0219; G06F 15/18

(57) ABSTRACT

Embodiments of the disclosure are directed to systems, methods, and devices for monitoring inertial data from a player swinging a piece of sports equipment. In embodiments, a monitoring device for monitoring swing metrics. The monitoring device includes an inertial measurement unit (IMU) implemented at least partially in hardware to sense inertial data corresponding to a swing of a piece of sports equipment; a processor implemented at least partially in hardware to extrapolate, based on the inertial data, one or more swing parameters associated with the swing of the piece of sports equipment; and a transceiver implemented at least partially in hardware to transmit the one or more swing parameters to a remote device across a wireless connection.

### TECHNICAL FIELD

This disclosure pertains to sensors for monitoring player performance, and more particularly, to monitoring player performance and providing analyses of player performance using on-board processing techniques, on-the-fly integration of feedback, and machine learning.

### BACKGROUND

On-board sensors for swing metrics analysis transmit raw data to processors that can process the draw data to draw conclusions about the swing and project potential results of the swing. Other sensors, such as high speed cameras and video can also be used as a mode of acquiring data for analysis. High speech cameras and video, however, are impractical for integration to a sporting equipment or player so as to avoid altering normal playing environments.

### BRIEF DESCRIPTION OF THE DRAWINGS

FIG. 1 is a schematic block diagram of a system 100 for monitoring player performance in accordance with embodiments of the present disclosure.

FIG. 2 is a schematic block diagram of an example monitoring device for monitoring player performance in accordance with embodiments of the present disclosure.

FIG. 3 is a schematic block diagram of another example monitoring device for monitoring player performance in accordance with embodiments of the present disclosure.

FIG. 4 is a process flow diagram for an example embodiment of monitoring player performance in accordance with embodiments of the present disclosure.

FIG. 5 is a process flow diagram for another example embodiment of monitoring player performance in accordance with embodiments of the present disclosure.

FIG. 6 is a process flow diagram for an example embodiment of processing player performance in accordance with embodiments of the present disclosure.

FIG. 7 is an example illustration of a processor according to an embodiment of the present disclosure.

FIG. 8 is a schematic block diagram of a mobile device in accordance with embodiments of the present disclosure.

FIG. 9 is a schematic block diagram of a computing system according to an embodiment of the present disclosure.

FIG. 10 is a process flow diagram for providing feedback to a monitoring device using a graphical user interface on a remote device in accordance with embodiments of the present disclosure.

### DETAILED DESCRIPTION

This disclosure involves a device that can be connected to a piece of sporting equipment, such as a bat, club, racquet, or player, that includes on-board sensors, processors, and communications systems. The on-board sensors can detect motion of the sporting equipment or player. An on-board processor can process the sensor data using machine learning to draw conclusions about what the sensor data means. The device can include a communications interface to transmit information to other devices and to receive feedback about conclusions drawn about the sensor data. The monitoring device can use machine learning as well as the feedback data to learn and improve on drawing conclusions about sensor data. The machine learning can include an initial training followed by on-the-fly learning based on feedback information.

The device can also include a memory for storing information that supports machine learning. The memory can also store information about the player, the player's equipment, default information, calibration information, etc. On-board machine learning and on-board storage of contextual data can allow the device to output contextual information about the swing that provides predicted meaning about the swing, instead of just outputting raw data. Raw data by itself may not provide much insight into the player performance, but including contextual information, by itself or along with player performance metrics, can provide more insight into the player performance.

The disclosure describes the use of sensors placed in wearable gloves and/or the sports equipment such as a cricket bat, baseball bat, golf club, tennis racquet, etc., and associated data analysis to calculate specific parameters. The disclosure addresses the problem of getting objective and quantitative data of player performance in sports that involves using a bat/racquet, while also decreasing transmission bandwidths, power consumption, complexity of transmission protocols, and increasing speed of processing and updating information.

The data and its associated analysis also helps increase audience interest as well as feedback to the player. The disclosure adds another dimension and modality of data to increase complementarity of data collected as well as increase the accuracy and the number of parameters from the analysis.

The basic principle is to incorporate simple sensors such as accelerometers & gyro meters that are non-intrusive, small and light weight to obtain data about the motion and the spatial coordinates of the racquet/bat. The data can then be analyzed to generate game parameters and statistics, player performance and comparison within and across games.

There are three use cases that are potential targets with this technology platform:

1. Commentator data support during broadcast—Additional data from the player standpoint can help a commentator compare as well as comment on. For example, player X has hit a great shot and has fantastic bat speed with minimal backlift that generates immense power.

2. Audience view enhancement—Actual data that is presented on the broadcast that gives readily understandable bit-sized information of interest. For example, comparison of bat speeds of top batsman and shots of interest.

3. Coaching—Parameters of interest during coaching and comparison to “ideal” parameters of top players. Parameters could be used for coaching overall hitting performance as well as for specific strokes.

More details are provided in the following description and accompanying figures, as well as in the claims.

The monitoring device can include sufficient processing to perform complex calculations of raw inertial data to extrapolate one or more swing parameters associated with a swing of a bat or club or racquet etc. The monitoring device can transmit the swing parameters instead of the raw data. By performing processing on the monitoring device, the amount of information transmitted and the required bandwidth can be reduced, and the transmission speed can be increased. The result is that real-time swing analyses can be provided to multiple users quickly, which can enhance the spectator experience and enhance commentator feedback to viewers. Similarly, spectators can receive swing parameter information while watching the game to get more information about how players approach certain situations, how well they execute swings, what the actual results of their intended swings are, etc.

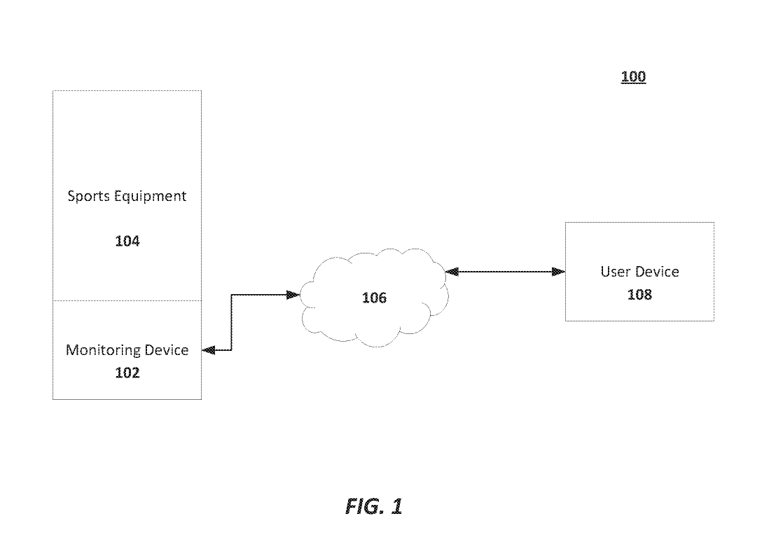

FIG. 1 is a schematic block diagram of a system 100 for monitoring player performance in accordance with embodiments of the present disclosure. The system 100 includes a. monitoring device 102 that can be attached to or integrated into a sports tool 104. The monitoring device 102 can be a puck or knob or button-shaped device that attaches to the end of a bat or club or can attach to a glove worn by the player. The sports tool 104 can include a bat, such as a cricket bat, baseball bat, softball bat, etc., a golf club, a racquet, such as tennis racquet, badminton racquet, squash racquet, racquetball racquet, etc.

The monitoring device 102 can include a communications interface, such as a. transceiver, to transmit and receive information from across a network 106. The monitoring device 102 transmit and receive information across network 106 from a user device 108 (or more than one user devices). The user device 108 can include a user interface to view player performance information and enter information as feedback to sensor-provided data. The user device 108 includes a network interface and at least one application programming interface to interface with the monitoring device across the network 106.

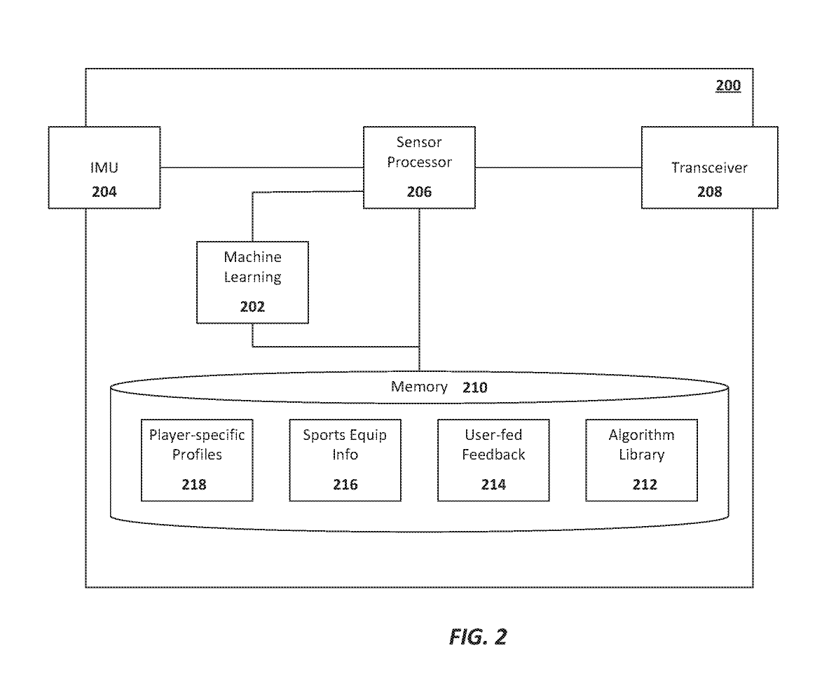

FIG. 2 is a schematic block diagram of an example monitoring device 200 for monitoring player performance in accordance with embodiments of the present disclosure. Monitoring device 200 may be a “puck” or “disc” or other shape that has a small form factor so that it is possible to mount the monitoring device 200 either directly to the non-hitting surface of the sporting equipment or on a players hand and/or body without interfering with the player's ability to swing naturally. The monitoring device 200 is configured to gather, analyze and transmit sensor data, such as data from inertial measurement unit (IMU) 204. The monitoring device can include a printed circuit board that includes a machine learning processor 202, an IMU 204, a transceiver 208, supporting hardware, such as sensor processor 206, memory 210, and a battery (not shown).

The monitoring device 200 can include an IMU 204. IMU 204 can be implemented in hardware, software, or a combination of hardware or software. The IMU 204 can include an accelerometer, gyroscope, magnetometer, or any other inertial measuring device. The IMU 204 can also include a combination of one or more of the above listed sensors. By using more than one sensor, the monitoring device 200 can “fuse” data from multiple sensors to extrapolate more information than if only one sensor were used. Sensor fusion from multiple inertial sensors can increase the accuracy of the sensory information and swing parameters.

The IMU 204 can output raw sensor data to the sensor processor 206: Sensor processor 206 can be implemented in hardware, software, or a combination of hardware or software. Sensor processor 206 can receive the raw sensor data from IMU 204 and translate the raw sensor data into a format that is understandable. For example, the sensor processor 206 can receive raw acceleration information and translate the raw acceleration information into a numeric value with units represented by bits of data.

The sensor processor 206 can include one or more processors implemented in hardware, software, or a combination of hardware and software. The sensor processor 206 can make use of a library 212 of newly developed, application-specific data analysis algorithms to recover relevant swing parameters from the raw data collected by the IMU 204. These algorithms leverage concepts from computational physics and mathematical simulation to detect swings in general or sport-specific swings amidst the other activities commonly experienced by the sensor (transport to and from the grounds, preparation for swing, etc.)). Once a swing is detected, the algorithms can recover relevant parameters from the swing. These parameters specifically can include: maximum and average speeds achieved by the tip of the sports equipment, maximum and average speeds achieved by the hands of the player, the length of the arc traversed by the tip of the sports equipment (segmented into the windup and follow through), and a variety of alignment measurements of the sports equipment face as the swing progresses.

Machine learning 202 can make use of these parameters to directly correlate to the quality of the swing based on training and feedback, and can be used to quantify and qualify the skill of the player, the predicted results of the swing, and suggest areas for improvement.

The first step in the algorithms is to calibrate the sensors relative to true magnetic north and gravity. After that, the data from all sensors and all three axes from accelerometer, magnetometer and gyro-meter are streamed. An Altitude Heading Reference System (AHRS) algorithm is used to convert the sensor raw data to Euler reference angles.

The accelerometer data is then smoothed looking for peaks above a certain threshold determined by empirical data.

Two sees (one on either side of the peak) of sensors and Euler angles data is buffered about the peak as determined. The Euler angles are used to determine the bottom most point of the swing at which point, the roll (i.e., the angle of the bat relative to the trajectory of the ball) is extracted.

To calculate backlift/backswing and follow through, the gyroscope measurements in the data stream are integrated until there is an inflection in the forward and the backward direction.

Using the determined backlift/backswing and follow through endpoints, the relevant data range for swing speed calculation is identified. For the hand and the bat speed calculations, the accelerometer data is integrated to determine linear speed and the gyroscope data is utilized to determine angular speed. By knowing the distance of the tip of the sporting equipment and the hand relative to the sensor placement, the angular speed can be converted to linear speeds of the hand and the bat. The measurements of the accelerometer and the gyrometer are then combined to determine the linear speed. The mean of these values through the identified swing is calculated as the average bat/hand speed. The maximum values are also returned as output parameters.

Swing parameters that can be extrapolated from sensor data include backlift/backswing, follow through, swing speed, momentum, swing arch, face angle, swing path, etc. Other player performance metrics can also be extrapolated from sensors in other locations, such as helmet sensors. A helmet sensor can extrapolate the motion or lack of motion of the player's head during a swing.

Drifts in the sensors hardware can build over time and yield offsets and inaccuracies, especially during integration into the device and sporting equipment. To compensate, only calibrated fractions of the accelerometer and gyro measurements are used, under the assumption that the drift is linear.

Monitoring device 200 can also include a memory 210. Memory 210 can store the algorithm library 212. Additionally, memory 210 can store sports equipment information 216, such as preprogrammed information about the bat or club or racquet. For example sports equipment information 216 can include a length and weight of a bat, a distance from the sensor location to the tip of the bat, the string tension of a tennis racquet, the strength of a golf club. etc. These preprogrammed sports equipment information can be used by the algorithms to determine player performance metrics. Player performance metrics can include, among other things, swing metrics, helmet metrics measuring motion/stillness of the head while facing the ball or swinging; impact measurements of ball hitting the helmet, lower body motion, such as step, hip rotation, etc.

Monitoring device 200 also includes a transceiver 208. Transceiver 208 can be implemented in hardware, software, or a combination of hardware and software. Transceiver 208 is configured to transmit processed sensor data to a remote device, such as a smartphone or tablet, that is running an application on a graphical user interface (GUI). A user of the remote device can provide feedback about the sensor data through the GUI and return the feedback to the monitoring device.

For example, based on a set of sensor data, processing, and in some cases, machine learning, the monitoring device can transmit that a player swung a bat at a certain speed, swing arc, backlift/backswing, etc., which suggests swing type A. A user can provide feedback that the swing resulted in something besides the expected results for swing type A. The user feedback can be received by the transceiver 208 and stored as user-fed feedback 214 in memory 210. The machine learning 202 can use the user-fed feedback 214 to correlate the specific swing parameters with the actual results observed so that the monitoring device can make more accurate predictions about the swings.

Transceiver 208 can include a Bluetooth transceiver, low frequency radio transceiver, or other type of transceiver, such as a Wifi transceiver, cellular transceiver, Internet of Things-type transceiver, etc. The mode of wireless transmission used depends on the distance to the recipient device and/or the amount of available bandwidth in the radio frequency.

In addition to the recovery of swing parameters, the monitoring device 200 can use machine learning 202 to identify the type of swing performed (such as a drive, a fly ball, a grounder, a fade, a draw, a server, a forehand, a backhand, etc.), as well as predicting swing results based on attempted swings. Machine learning processor 202 can be implemented in hardware, software, or a combination of hardware and software. The machine learning processor 202 can use feedback information from the user to make corrections or confirmations of how the monitoring device 200 correlates swing parameters with swing types, predicted swing results, or other predictions.

The player can also interact with the monitoring device 200 through an application on a remote device, such as a smartphone or tablet. The player can provide inputs thought a GUI about a swing the player is about to attempt. The monitoring system 200 can provide feedback through the GUI about whether the attempted swings have swing parameters that correlate to the attempted swing or with other swings or with nothing stored. The player can then provide feedback to the monitoring system 200 through the GUI about the results of a swing, The machine learning 202 can use the player-fed feedback to correlate the swing parameters with the results of the swing. This correlation can create a player-specific profile 218 that correlates player-specific swing parameters with various types of swings, etc.

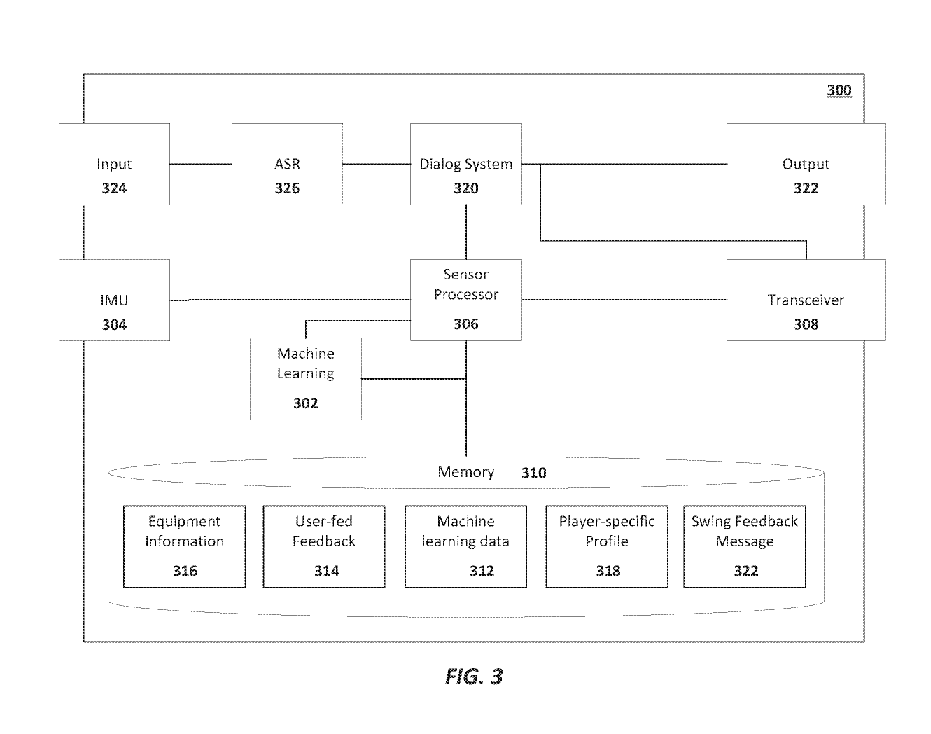

FIG. 3 is a schematic block diagram of another example monitoring device 300 for monitoring player performance in accordance with embodiments of the present disclosure. Monitoring device 300 shares similar features as monitoring device 200 described above. Monitoring device 300 includes a sensor processor 306, IMU 304, machine learning processor 302, transceiver 308, and memory 310, each of which are similar to that described in FIG. 2.

Monitoring device 300 includes a dialog system 320 for facilitating a dialog between the player or coach and the monitoring system 300. Monitoring device 300 also includes an input 324 such as a microphone or text-based input. The input 324 can provide the input to an automatic speech recognition (ASR) system 326 that translates the input into recognized text. The recognized text is provided to the dialog system 320. Dialog system 320 processes the received recognized text and interprets the intent based on the input. The dialog system 320 can identify a response to the input and provide the response to an output 322. Output 322 can include a speaker or text output; in some embodiments, the output 322 can include a transmitter, such as a Bluetooth transmitter that an output text or audible speech through a separate device.

In one example implementation, a player may start a dialog with the monitoring device 300 by telling the monitoring device 300 that the player is about to attempt a specific swing type, such as a fade in golf. The dialog system 320 can interpret the input speech and signal to the sensor processor 306 that the player is about to attempt a fade. As the player takes a practice swing, for example, the sensor processor 306 and machine learning 302 can process sensor data from the IMU to predict whether the practice swing would result in a fade. The sensor processor 306 can relay that information to the dialog system 320. Dialog system 320 can create a message or can use a predetermined swing feedback message 322 to provide immediate swing feedback to the player and/or coach. If the practice swing does not have the characteristics (i.e., swing parameters) associated with a fade, the dialog system can inform the player that 1) the swing is not a fade and/or 2) what the swing is predicted to be (e.g., a draw, straight flight, hook, slice, etc.). The player can continue taking practice swings until the dialog system 320 confirms that the swing has the characteristics of a fade. The player can then take an actual swing trying to repeat the swing of a fade.

The monitoring system 300 can then track swing parameters of the actual swing. The player can provide direct feedback through the dialog system 320 of whether the swing resulted in a fade as intended or resulted in something else. The dialog system 320 can provide feedback to the player of whether the actual swing had characteristics of a fade or something else using the tracked swing parameters of the actual swing. The machine learning 302 can use the player-fed feedback through the dialog system 320 to correlate the swing parameters with the results of the swing. This correlation can create a player-specific profile 318 that correlates player-specific swing parameters with various types of swings, etc.

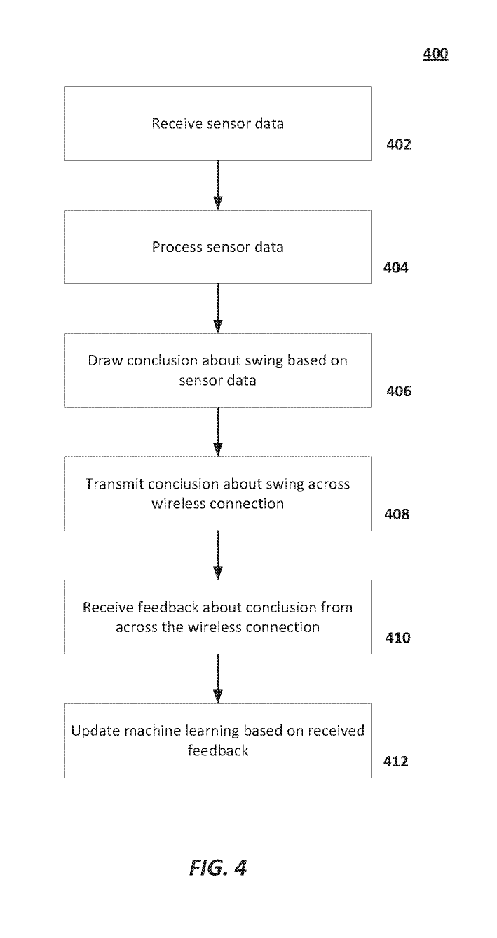

FIG. 4 is a process flow diagram 400 for an example embodiment of monitoring player performance in accordance with embodiments of the present disclosure. Upon detecting an intentional swing, the inertial measurement unit (IMU) can receive sensor data (402). The sensor data can be processed to determine swing parameters (404). A conclusion can be reached about the swing parameters based on correlating swing parameters with known swing information and through machine learning techniques (406). The conclusions about the swing can be transmitted to a user across a wireless connection (408). Feedback from the user can be received from the wireless connection (410). The feedback can be used to update the machine learning (412).

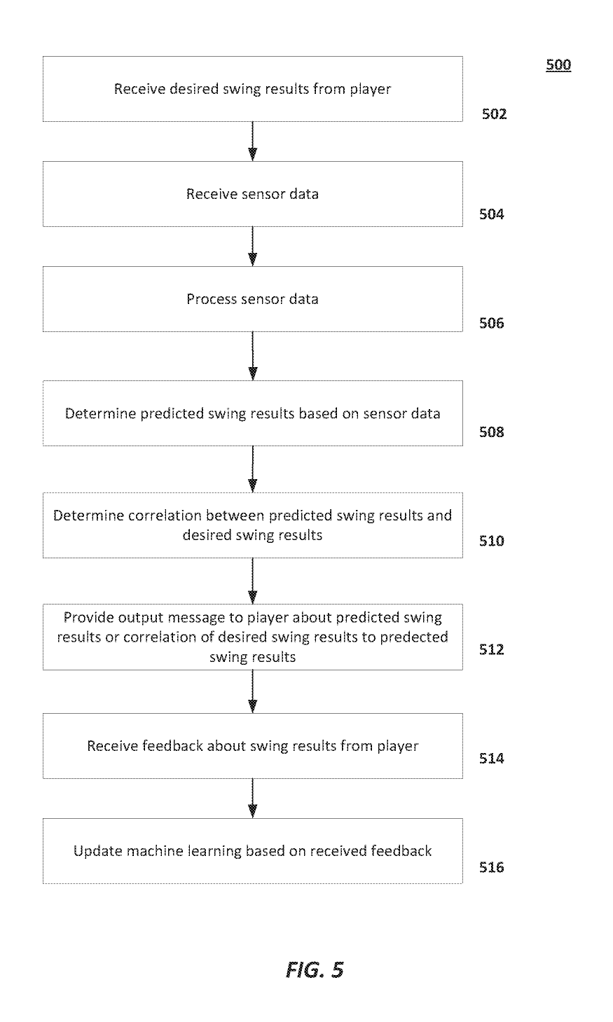

FIG. 5 is a process flow diagram 500 for another example embodiment of monitoring player performance in accordance with embodiments of the present disclosure. A player can provide desired swing results or an intend swing type to the monitoring device (502). The player can provide the swing information through a GUI on a remote device or via a dialog system. Upon detecting an intentional swing, the inertial measurement unit (IMU) can receive sensor data (504). The sensor data can be processed to determine swing parameters (506). A conclusion can be reached about the swing parameters based on correlating swing parameters with known swing information and through machine learning techniques (508). The conclusions about the swing parameters can be compared against the intended swing information provided by the player (510). The correlation between the swing parameters detected and the intended swing can be provided to the player (512). The correlation information can be provided through a GUI on a remote device or through a dialog system or both. Feedback from the user can be received from the wireless connection (514). The feedback The feedback can be used to update the machine learning (516).

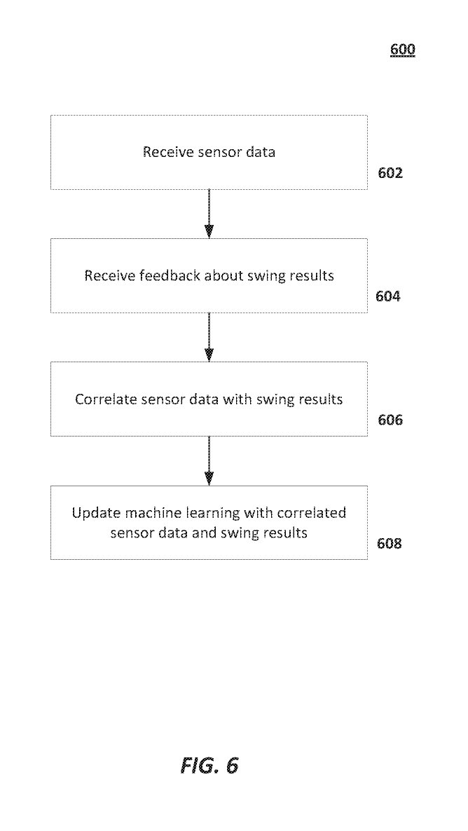

FIG. 6 is a process flow diagram 600 for an example embodiment of processing player performance feedback in accordance with embodiments of the present disclosure. The monitoring device can receive sensor data (602). The monitoring system can receive feedback about the actual swing results (604). The monitoring can correlate the sensor data with the received feedback about the actual swing results (606). The machine learning can update using the correlated information from the feedback and the sensor data (608).

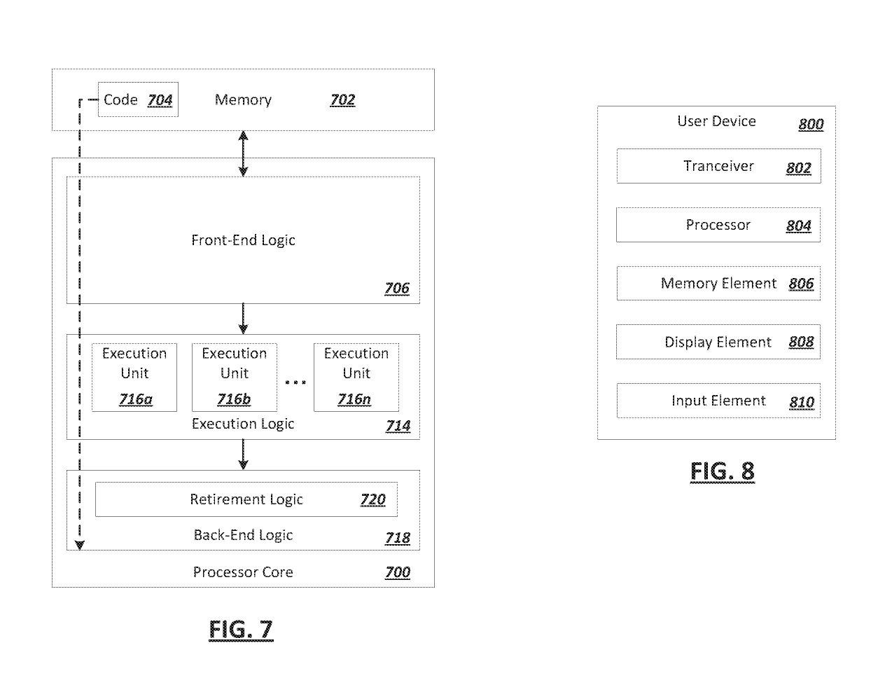

FIGS. 7-8 are block diagrams of exemplary computer architectures that may be used in accordance with embodiments disclosed herein. Other computer architecture designs known in the art for processors, mobile devices, and computing systems may also be used. Generally, suitable computer architectures for embodiments disclosed herein can include, but are not limited to, configurations illustrated in FIGS. 7-8.

FIG. 7 is an example illustration of a processor according to an embodiment. Processor 700 is an example of a type of hardware device that can be used in connection with the implementations above.

Processor 700 may be any type of processor, such as a microprocessor, an embedded processor, a digital signal processor (DSP), a network processor, a multi-core processor, a single core processor, or other device to execute code. Although only one processor 700 is illustrated in FIG. 7, a processing element may alternatively include more than one of processor 700 illustrated in FIG. 7. Processor 700 may be a single-threaded core or, for at least one embodiment, the processor 700 may be multi-threaded in that it may include more than one hardware thread context (or “logical processor”) per core.

FIG. 7 also illustrates a memory 702 coupled to processor 700 in accordance with an embodiment. Memory 702 may be any of a wide variety of memories (including various layers of memory hierarchy) as are known or otherwise available to those of skill in the art. Such memory elements can include, but are not limited to, random access memory (RAM), read only memory (ROM), logic blocks of a field programmable gate array (FPGA), erasable programmable read only memory (EPROM), and electrically erasable programmable ROM (EEPROM).

Processor 700 can execute any type of instructions associated with algorithms, processes, or operations detailed herein. Generally, processor 700 can transform an element or an article (e.g., data.) from one state or thing to another state or thing.

Code 704, which may be one or more instructions to be executed by processor 700, may be stored in memory 702, or may be stored in software, hardware, firmware, or any suitable combination thereof, or in any other internal or external component, device, element, or object where appropriate and based on particular needs. In one example, processor 700 can follow a program sequence of instructions indicated by code 704. Each instruction enters a front-end logic 706 and is processed by one or more decoders 708. The decoder may generate, as its output, a micro operation such as a fixed width micro operation in a predefined format, or may generate other instructions, microinstructions, or control signals that reflect the original code instruction. Front-end logic 706 also includes register renaming logic 610 and scheduling logic 712, which generally allocate resources and queue the operation corresponding to the instruction for execution.

Processor 700 can also include execution logic 714 having a set of execution units 716a, 716b, 716n, etc. Some embodiments may include a number of execution units dedicated to specific functions or sets of functions. Other embodiments may include only one execution unit or one execution unit that can perform a particular function. Execution logic 714 performs the operations specified by code instructions.

After completion of execution of the operations specified by the code instructions, back-end logic 718 can retire the instructions of code 704. In one embodiment, processor 700 allows out of order execution but requires in order retirement of instructions. Retirement logic 720 may take a variety of known forms (e.g., re-order buffers or the like). In this manner, processor 700 is transformed during execution of code 704, at least in terms of the output generated h the decoder, hardware registers and tables utilized by register renaming logic 710, and any registers (not shown) modified by execution logic 714.

Although not shown in FIG. 7, a processing element may include other elements on a chip with processor 700. For example, a processing element may include memory control logic along with processor 700. The processing element may include I/O control logic and/or may include I/O control logic integrated with memory control logic. The processing element may also include one or more caches. In some embodiments, non-volatile memory (such as flash memory or fuses) may also be included on the chip with processor 700.

Referring now to FIG. 7 a block diagram is illustrated of an example mobile device 800. Mobile device 800 is an example of a possible computing system (e.g., a host or endpoint device) of the examples and implementations described herein. In an embodiment, mobile device 800 operates as a transmitter and a receiver of wireless communications signals. Specifically, in one example, mobile device 800 may be capable of both transmitting and receiving cellular network voice and data mobile services. Mobile services include such functionality as full Internet access, downloadable and streaming video content, as well as voice telephone communications.

Mobile device 800 may correspond to a conventional wireless or cellular portable telephone, such as a handset that is capable of receiving “3G”, or “third generation” cellular services. In another example, mobile device 800 may be capable of transmitting and receiving “4G” mobile services as well, or any other mobile service.

Examples of devices that can correspond to mobile device 800 include cellular telephone handsets and smartphones, such as those capable of Internet access, email, and instant messaging communications, and portable video receiving and display devices, along with the capability of supporting telephone services. It is contemplated that those skilled in the art having reference to this specification will readily comprehend the nature of modem smartphones and telephone handset devices and systems suitable for implementation of the different aspects of this disclosure as described herein. As such, the architecture of mobile device 800 illustrated in FIG. 8 is presented at a relatively high level. Nevertheless, it is contemplated that modifications and alternatives to this architecture may be made and will be apparent to the reader, such modifications and alternatives contemplated to be within the scope of this description.

In an aspect of this disclosure, mobile device 800 includes a transceiver 802, which is connected to and in communication with an antenna. Transceiver 802 may be a radio frequency transceiver. Also, wireless signals may be transmitted and received via transceiver 802. Transceiver 802 may be constructed, for example, to include analog and digital radio frequency (RF) ‘front end’ functionality, circuitry for converting RF signals to a baseband frequency, via an intermediate frequency (IF) if desired, analog and digital filtering, and other conventional circuitry useful for carrying out wireless communications over modem cellular frequencies, for example, those suited for 3G or 4G communications. Transceiver 802 is connected to a processor 804, which may perform the bulk of the digital signal processing of signals to be communicated and signals received, at the baseband frequency. Processor 804 can provide a graphics interface to a display element 808, for the display of text, graphics, and video to a user, as well as an input element 710 for accepting inputs from users, such as a touchpad, keypad, roller mouse, and other examples. Processor 804 may include an embodiment such as shown and described with reference to processor 700 of FIG. 7.

In an aspect of this disclosure, processor 804 may be a processor that can execute any type of instructions to achieve the functionality and operations as detailed herein. Processor 804 may also be coupled to a memory element 806 for storing information and data used in operations performed using the processor 804. Additional details of an example processor 804 and memory element 806 are subsequently described herein. In an example embodiment, mobile device 800 may be designed with a system-on-a-chip (SoC) architecture, which integrates many or all components of the mobile device into a single chip, in at least some embodiments.

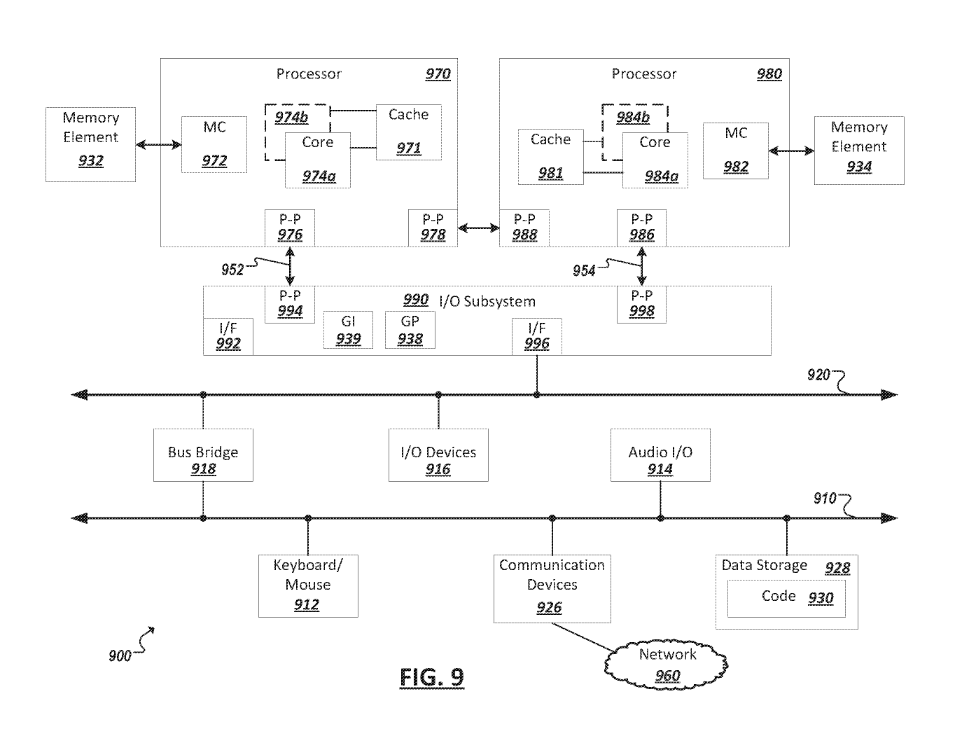

FIG. 9 is a schematic block diagram of a computing system 900 according to an embodiment. In particular, FIG. 9 shows a system where processors, memory, and input/output devices are interconnected by a number of point-to-point interfaces. Generally, one or more of the computing systems described herein may be configured in the same or similar manner as computing system 900.

Processors 970 and 980 may also each include integrated memory controller logic (MC) 972 and 982 to communicate with memory elements 932 and 934. In alternative embodiments, memory controller logic 972 and 982 may be discrete logic separate from processors 970 and 980. Memory elements 932 and/or 934 may store various data to be used by processors 970 and 980 in achieving operations and functionality outlined herein.

Processors 970 and 980 may be any type of processor, such as those discussed in connection with other figures. Processors 970 and 980 may exchange data via a point-to-point (PtP) interface 950 using point-to-point interface circuits 978 and 988, respectively. Processors 970 and 980 may each exchange data with a chipset 990 via individual point-to-point interfaces 952 and 954 using point-to-point interface circuits 976, 986, 994, and 998. Chipset 990 may also exchange data with a high-performance graphics circuit 938 via a high-performance graphics interface 939, using an interface circuit 992, which could be a PtP interface circuit. In alternative embodiments, any or all of the PtP links illustrated in FIG. 9 could be implemented as a multi-drop bus rather than a PtP link.

Chipset 990 may be in communication with a bus 920 via an interface circuit 996. Bus 920 may have one or more devices that communicate over it, such as a bus bridge 918 and I/O devices 916. Via a bus 910, bus bridge 918 may he in communication with other devices such as a keyboard/mouse 912 (or other input devices such as a touch screen, trackball, etc.), communication devices 926 (such as modems, network interface devices, or other types of communication devices that may communicate through a computer network 960), audio I/O devices 914, and/or a data storage device 928. Data storage device 928 may store code 930, which may be executed by processors 970 and/or 980. In alternative embodiments, any portions of the bus architectures could be implemented with one or more PtP links.

The computer system depicted in FIG. 9 is a schematic illustration of an embodiment of a computing system that may be utilized to implement various embodiments discussed herein. It will be appreciated that various components of the system depicted in FIG. 9 may be combined in a system-on-a-chip (SoC) architecture or in any other suitable configuration capable of achieving the functionality and features of examples and implementations provided herein.

FIG. 9 is a process flow diagram 900 for dynamically determining a threshold noise level. The dialog system can store failed attempts to interpret recognized speech (902). The dialog system can also store coincidental background noise levels (i.e., background noise that may be received at the same time as the audible speech signal) (904). The dialog system can correlate failed attempts to interpret recognized text with the corresponding background noise (906). For example, the dialog system can consider whether the recognized text was interpreted correctly, the number of times the user had to repeat him/herself, etc. The dialog system can determine a threshold noise level for background noise level for determining whether the noise level is too high for the ASR to accurately recognize audible speech (908).

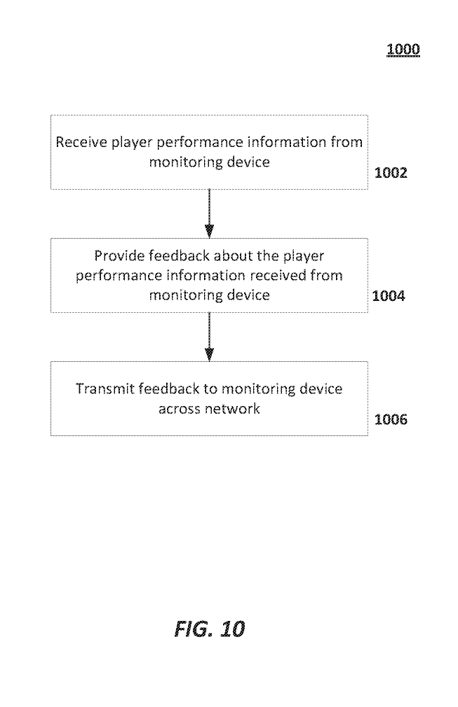

FIG. 10 is a process flow diagram 1000 for providing feedback to a monitoring device using a graphical user interface on a remote device in accordance with embodiments of the present disclosure. The remote device can be a smartphone or tablet or other connected device. The remote device can receive player performance information from the monitoring device from a wireless connection (1002). The user can provide feedback about the player performance information to the monitoring device across the wireless connection (1004). The feedback can include actual results about a swing or other feedback. The user can transmit the feedback through the remote device across the wireless connection to the monitoring device (1006).

In some embodiments, a tablet or smartphone application can be used. The application can provide a graphical user interface (GUI) for viewing swing parameters and other information. The application receives the data from the monitoring device. Based upon the data stream the application contains algorithms that detect a swing motion. Upon detection of a swing the data around the event is buffered and then statistics are calculated based on the IMU data. Those statistics may include hand speed, bat speed, follow through, back lift, roll, straight, timing sweet spot, and swing classification. Additionally the IMU data can be used to create a virtual representation of the swing.

Example 1 a monitoring device for monitoring swing metrics, the monitoring device comprising an inertial measurement unit (IMU) implemented at least partially in hardware to sense inertial data corresponding to a swing of a piece of sports equipment; a processor implemented at least partially in hardware to extrapolate, based on the inertial data, one or more swing parameters associated with the swing of the piece of sports equipment; and a transceiver implemented at least partially in hardware to transmit the one or more swing parameters to a remote device across a wireless connection.

Example 2 may include the subject matter of example 1, wherein the transceiver is configured to receive feedback information from the remote device across the wireless connection.

Example 3 may include the subject matter of any of examples 1 or 2, further comprising a machine learning processor implemented at least partially in hardware to correlate feedback information with swing parameters and received inertial data.

Example 4 may include the subject matter of example 3, wherein the machine learning processor is configured to use feedback information to update machine learning libraries.

Example 5 may include the subject matter of any of examples 1-4, wherein the IMU comprises one or a combination of an accelerometer, gyrometer, or magnetometer,

Example 6 may include the subject matter of any of examples 1-5, wherein the transceiver comprises one or more of a Bluetooth transceiver, Will transceiver, radio frequency transceiver, or cellular transceiver.

Example 7 may include the subject matter of any of examples 1-6, wherein the monitoring device is configured to attached to the piece of sporting equipment, wherein the sporting equipment comprises one of a cricket bat, a baseball bat, a softball bat, a golf club, or a racquet.

Example 8 is a method for monitoring player performance, the method comprising receiving inertial data at an inertial measurement unit; processing the inertial data to determine a swing parameter; transmitting the swing parameter to a remote device across a wireless connection.

Example 9 may include the subject matter of example 8, further comprising receiving feedback information from the remote device from across the wireless connection.

Example 10 may include the subject matter of example 8, further comprising receiving an input from a player, the input indicating an intended swing; receiving inertial data associated with an actual swing; processing the inertial data associated with the actual swing; determining whether the actual swing correlates to the intended swing based on the inertial data.

Example 11 may include the subject matter of example 10, further comprising receiving feedback information from the player about a result of the actual swing; determining whether the result of the actual swing correlates to the intended swing.

Example 12 may include the subject matter of example 11, further comprising updating a machine learning library based on the feedback information.

Example 13 may include the subject matter of example 11, further comprising creating a player-specific profile based on correlating the inertial data with the feedback information about the result of the actual swing, the player-specific profile comprising player-specific swing parameters that correlate to swing results.

Example 14 may include the subject matter of example 10, further comprising providing an output to the player informing the player whether the actual swing correlates to the intended swing.

Example 15 may include the subject matter of any of examples 8-14, wherein the IMU comprises one or a combination of an accelerometer, gyrometer, or magnetometer.

Example 16 may include the subject matter of any of examples 8-15. wherein the transceiver comprises one or more of a Bluetooth transceiver, Wifi transceiver, radio frequency transceiver, or cellular transceiver.

Example 17 is a system comprising a monitoring device comprising: an inertial measurement unit (IMU) implemented at least partially in hardware to sense inertial data corresponding to a swing of a piece of sports equipment, a processor implemented at least partially in hardware to extrapolate, based on the inertial data, one or more swing parameters associated with the swing of the piece of sports equipment, a transceiver implemented at least partially in hardware to transmit the one or more swing parameters to a remote device across a wireless connection. The system may also include a sporting equipment, the monitoring device attached to the sporting equipment.

Example 18 may include the subject matter of example 17, wherein the transceiver is configured to receive feedback information from the remote device across the wireless connection.

Example 19 may include the subject matter of any of examples 17 or 18, further comprising a machine learning processor implemented at least partially in hardware to correlate feedback information with swing parameters and received inertial data.

Example 20 may include the subject matter of example 19, wherein the machine learning processor is configured to use feedback information to update machine learning libraries.

Example 21 may include the subject matter of any of examples 17-20, wherein the IMU comprises one or a combination of an accelerometer, gyrometer, or magnetometer.

Example 22 may include the subject matter of any of examples 17-21, wherein the transceiver comprises one or more of a Bluetooth transceiver, Wifi transceiver, radio frequency transceiver, or cellular transceiver.

Example 23 may include the subject matter of any of examples 17-22, wherein the monitoring device is configured to attached to the piece of sporting equipment, wherein the sporting equipment comprises one of a cricket bat, a baseball bat, a softball bat, a golf club, or a racquet.

Example 24 is a computer program product tangibly embodied on non-transient computer readable media, the computer program product comprising instructions operable when executed to receive swing parameter information about a player's swing from a monitoring device from across a wireless connection; receive user-supplied feedback information about the swing parameter; and transmit the feedback information to the monitoring device across the wireless connection.

Example 25 may include the subject matter of example 24, further comprising creating a virtual representation of the player's swing using the swing parameter information received from the monitoring device.

Although this disclosure has been described in terms of certain implementations and generally associated methods, alterations and permutations of these implementations and methods will be apparent to those skilled in the art. For example, the actions described herein can be performed in a different order than as described and still achieve the desirable results. As one example, the processes depicted in the accompanying figures do not necessarily require the particular order shown, or sequential order, to achieve the desired results. In certain implementations, multitasking and parallel processing may be advantageous. Additionally, other user interface layouts and functionality can be supported. Other variations are within the scope of the following claims.

While this specification contains many specific implementation details, these should not be construed as limitations on the scope of any disclosures or of what may be claimed, but rather as descriptions of features specific to particular embodiments of particular disclosures. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

1. A monitoring device for monitoring swing performance metrics, the monitoring device comprising:
an inertial measurement unit (IMU) implemented at least partially in hardware to sense inertial data corresponding to a motion of sporting equipment;
a processor implemented at least partially in hardware to extrapolate, based on the inertial data, one or more performance parameters associated with the motion of the sporting equipment; and
a transceiver implemented at least partially in hardware to transmit the one or more performance parameters to a remote device across a wireless connection.

2. The monitoring device of claim 1, wherein the transceiver is configured to receive feedback information from the remote device across the wireless connection.

3. The monitoring device of claim 1, further comprising a machine learning processor implemented at least partially in hardware to correlate feedback information with swing parameters and received inertial data.

4. The monitoring device of claim 3, wherein the machine learning processor is configured to use feedback information to update machine learning libraries.

5. The monitoring device of claim 1, wherein the IMU comprises one or a combination of an accelerometer, gyrometer, or magnetometer.

6. The monitoring device of claim 1, wherein the transceiver comprises one or more of a Bluetooth transceiver, Wifi transceiver, radio frequency transceiver, or cellular transceiver.

7. The monitoring device of claim 1, wherein the monitoring device is configured to attached to the sporting equipment, wherein the sporting equipment comprises one of a cricket bat, a baseball bat, a softball bat, a golf club, or a racquet, or a helmet, a glove, a pad, or a uniform.

8. A method for monitoring player performance, the method comprising:
receiving inertial data at an inertial measurement unit (IMU);
processing the inertial data to determine a performance parameter; and
transmitting the performance parameter to a remote device across a wireless connection.

9. The method of claim 8, further comprising receiving feedback information from the remote device from across the wireless connection.

10. The method of claim 8, further comprising:
receiving an input from a player, the input indicating an intended swing;
receiving inertial data associated with an actual swing;
processing the inertial data associated with the actual swing; and
determining whether the actual swing correlates to the intended swing based on the inertial data.

11. The method of claim 10, further comprising:
receiving feedback information from the player about a result of the actual swing; and
determining whether the result of the actual swing correlates to the intended swing.

12. The method of claim 11, further comprising updating a machine learning library based on the feedback information.

13. The method of claim 11, further comprising creating a player-specific profile based on correlating the inertial data with the feedback information about the result of the actual swing, the player-specific profile comprising player-specific swing parameters that correlate to swing results.

14. The method of claim 10, further comprising providing an output to the player informing the player whether the actual swing correlates to the intended swing.

15. The method of claim 8, wherein the IMU comprises one or a combination of an accelerometer, gyrometer, or magnetometer.

16. The method of claim 8, wherein the transmitting comprises utilizing one or more of a Bluetooth transceiver, Wifi transceiver, radio frequency transceiver, or cellular transceiver.

17. A system comprising:
a monitoring device comprising:
an inertial measurement unit (IMU) implemented at least partially in hardware to sense inertial data corresponding to a motion of a piece of sports equipment,
a processor implemented at least partially in hardware to extrapolate, based on the inertial data, one or more performance parameters associated with the motion of the piece of sports equipment,
a transceiver implemented at least partially in hardware to transmit the one or more performance parameters to a remote device across a wireless connection; and
a sporting equipment, the monitoring device attached to the sporting equipment.

18. The system of claim 17, wherein the transceiver is configured to receive feedback information from the remote device across the wireless connection.

19. The system of claim 17, further comprising a machine learning processor implemented at least partially in hardware to correlate feedback information with swing parameters and received inertial data.

20. The system of claim 19, wherein the machine learning processor is configured to use feedback information to update machine learning libraries.

21. The system of claim 17, wherein the IMU comprises one or a combination of an accelerometer, gyrometer, or magnetometer.

22. The system of claim 17, wherein the transceiver comprises one or more of a Bluetooth transceiver, Wifi transceiver, radio frequency transceiver, or cellular transceiver.

23. The system of claim 17, wherein the monitoring device is configured to attached to the piece of sporting equipment, wherein the sporting equipment comprises one of a cricket bat, a baseball bat, a softball bat, a golf club, or a racquet.

24. A computer program product tangibly embodied on non-transient computer readable media, the computer program product comprising instructions operable when executed to:
receive swing parameter information about a player's swing from a monitoring device from across a wireless connection;
receive user-supplied feedback information about the swing parameter information; and
transmit the feedback information to the monitoring device across the wireless connection.

25. The computer program product of claim 24, further comprising creating a virtual representation of the player's swing using the swing parameter information received from the monitoring device.
